SEQUENCE LISTING (18 sequences)

SEQ ID NO: 1 - protein, 8 aa, Artificial Sequence
  peptide selectively binding to graphitic materials
  VARIANT (1)..(1): X is D, E, N, or Q
  VARIANT (3)..(3): X is W, Y, F or H
  VARIANT (6)..(6): X is D, E, N or Q
  VARIANT (7)..(7): X is I, L, or V
XSXAAXXP

SEQ ID NO: 2 - protein, 8 aa, ARTIFICIAL SEQUENCE
  peptide selectively binding to graphitic materials
  VARIANT (1)..(1): X is D, E, N, or Q
  VARIANT (2)..(2): X is D, E, N, or Q
  VARIANT (4)..(4): X is I, L, or V
  VARIANT (5)..(5): X is D, E, N, or Q
  VARIANT (7)..(7): X is I, L, or V
XXPXXAXP

SEQ ID NO: 3 - protein, 7 aa, Artificial Sequence
  peptide selectively binding to graphitic materials
  VARIANT (2)..(2): X is W, Y, F, or H
  VARIANT (5)..(5): X is D, E, N, or Q
  VARIANT (6)..(6): X is I, L, or V
SXAAXXP

SEQ ID NO: 4 - protein, 7 aa, Artificial Sequence
  peptide selectively binding to graphitic materials
  VARIANT (1)..(1): X is D, E, N, or Q
  VARIANT (3)..(3): X is I, L, or V
  VARIANT (4)..(4): X is D, E, N, or Q
  VARIANT (6)..(6): X is I, L, or V
XPXXAXP

SEQ ID NO: 5 - protein, 8 aa, Artificial Sequence
  peptide selectively binding to graphitic materials
DSWAADIP

SEQ ID NO: 6 - protein, 8 aa, Artificial Sequence
  peptide selectively binding to graphitic materials
DNPIQAVP

SEQ ID NO: 7 - protein, 7 aa, Artificial Sequence
  peptide selectively binding to graphitic materials
SWAADIP

SEQ ID NO: 8 - protein, 7 aa, Artificial Sequence
  peptide selectively binding to graphitic materials
NPIQAVP

SEQ ID NO: 9 - protein, 12 aa, Artificial Sequence
  peptide selectively binding to graphitic materials
ADSWAADIPDPA

SEQ ID NO: 10 - protein, 27 aa, Artificial Sequence
  peptide selectively binding to graphitic material
ADSWAADIPDPAGGGADSWAADIPDPA

SEQ ID NO: 11 - protein, 33 aa, Artificial Sequence
  peptide selectively binding to graphitic material
ADSWAADIPDPAKAAGGGADSWAADIPDPAKAA

SEQ ID NO: 12 - protein, 7 aa, Artificial Sequence
  peptide selectively binding to graphitic materials
YYACAYY

SEQ ID NO: 13 - DNA, 7222 bp, Artificial Sequence
  cloning vector M13KE
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagtggtacctttctattctcactcggccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgttccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 14 - DNA, 41 bp, Artificial Sequence
  BamH I_SM_upper which is a primer used for site-directed mutation
aaggccgcttttgcgggatcctcaccctcagcagcgaaaga

SEQ ID NO: 15 - DNA, 41 bp, Artificial Sequence
  BamH I_SM_lower which is a primer used for site-directed mutation
tctttcgctgctgagggtgaggatcccgcaaaagcggcctt

SEQ ID NO: 16 - DNA, 90 bp, Artificial Sequence
  BamM13HK_P8_primer which is an extension primer used for preparation
ttaatggaaacttcctcatgaaaaagtctttagtcctcaaagcctctgtagccgttgctaccctcgttccgatgctgtctttcgctgctg

SEQ ID NO: 17 - DNA, 95 bp, Artificial Sequence
  M13HK_P8 which is a library oligonucleotide used for preparation
  misc_feature (1)..(95): n is a, g, c or t
  misc_feature (1)..(95): m is a or c
aaggccgcttttgcgggatccnnmnnmnnmnnmnnmnnmnnmncagcagcgaaagacagcatcggaacgagggtagcaacggctacagaggcttt

SEQ ID NO: 18 - protein, 50 aa, Artificial Sequence
  P8 protein of M13 phage
AEGDDPAKAAFNSLQASATEYIGYAWAMVVVIVGATIGIKLFKKFTSKAS

(19) United States
(12) Patent Application Publication
(10) Pub. No.: US 2020/0070403 A1
(43) Pub. Date: Mar. 5, 2020

(54) METHOD OF PRODUCING NANOPARTICLE DEVICE USING PRINT-ON HYDROGEL

(71) Applicant: KOREA INSTITUTE OF SCIENCE AND TECHNOLOGY, Seoul (KR)

(72) Inventors: Hyunjung YI, Seoul (KR); Tae Hyung KANG, Seoul (KR); Hochan CHANG, Seoul (KR); Ki-Young LEE, Seoul (KR)

(21) Appl. No.: 16/548,405

(22) Filed: Aug. 22, 2019

(30) Foreign Application Priority Data
Aug. 30, 2018 (KR) 10-2018-0103035

(51) Int. Cl. B29C 64/112; C07K 7/06; C09D 11/03; C01B 32/159; C12Q 1/00; B33Y 10/00; B33Y 70/00; B33Y 80/00

(52) U.S. Cl. CPC B29C 64/112; C07K 7/06; C09D 11/03; C01B 32/159; B29K 2105/0061; B33Y 10/00; B33Y 70/00; B33Y 80/00; C12Q 1/006

(57) ABSTRACT

Provided are a method of producing a nanoparticle device and a nanoparticle device. The method of producing a nanoparticle device may be economical due to use of a hydrogel, may be easy to design in terms of mass production processes, and may reduce manufacturing times to 1/100 to 1/10 of the technology of the related art. In addition, a nanoparticle device may be produced in various designs by stably realizing a 3D pattern and pattern stacking, and may have highly uniform nanoparticle dispersion and excellent electrical activity through the removal of a surfactant without damaging the pattern. The nanoparticle device produced according to the production method may have excellent electrical activity due to nanoparticle uniformity pattern accuracy and thus may be applied to pattern stacking which could not be implemented by methods of the related art.

### CROSS-REFERENCE TO RELATED APPLICATION

This application claims the benefit of Korean Patent Application No. 10-2018-0103035, filed on Aug. 30, 2018, in the Korean Intellectual Property Office, the disclosure of which is incorporated herein in its entirety by reference.

### SEQUENCE LISTING

This application includes a sequence listing submitted as an ASCII text file named 8F45696.TXT created Nov. 4, 2019 and of size 16 kB, which is incorporated by reference herein.

### BACKGROUND

### 1. Field

One or more embodiments relate to a method of producing a device including nanoparticles.

### 2. Description of Related Art

With the advent of the era of Internet of Things in which objects are interconnected with other objects or people, the role of wearable devices has been further emphasized. Accordingly, there has recently been a rapid increase in the demand for technology for flexible and high-performance materials and devices. To achieve such purposes, research has been conducted on various flexible and high-performance nanoparticle materials such as carbon nanotubes, graphene, and metal nanowires. Because these materials have very small forms on a nanoscale, there is a need to develop efficient manufacturing processes capable of precisely manufacturing devices in sizes of micrometers, millimeters, and centimeters for actual application of these materials to the devices.

Among various device manufacturing processes, particularly coating processes or printing processes such as screen printing and inkjet printing, solution processes enable mass production, allow various materials to be stacked, significantly decrease the number of processes, and reduce manufacturing costs by eliminating the waste of raw materials. Due to these properties, coating processes or printing processes have received attention as manufacturing processes for next-generation wearable devices.

However, nanoparticle materials such as carbon nanotubes, graphene, and metal nanowires exhibit low dispersion stability in water-based inks, and thus many problems arise in manufacturing of devices by printing. To improve dispersion stability, a method of applying a functional group to a nanomaterial by chemical treatment to produce a single-walled carbon nanotube ink has been reported. When a functional group is applied to carbon nanotubes by chemical treatment, the structure of the carbon nanotubes is deformed, thereby deteriorating the original electrical and electrochemical characteristics of the nanotubes.

Thus, in fields where electrical and electrochemical characteristics are important, a widely used method is the dispersal of nanoparticle materials such as carbon nanotubes or metal nanowires in water using a surfactant, to prepare a colloidal composition which is used as an ink. Examples of the surfactant may include sodium cholate, sodium dodecyl sulfate, and the like. Although surfactants are required to prepare colloidal solutions, they may act as foreign substances that interfere with the action of a device such as an electrode manufactured using a surfactant. For example, a surfactant present between printed carbon nanotubes may deteriorate electrical characteristics of a thin film and may partially dissolve and flow out when brought into contact with water again. Thus, washing with water (washing process) or chemical post processing such as acid-treatment has been used to remove surfactants included in nanoparticle materials. However, according to the above-described processes, printed carbon nanotubes may partially dissolve and flow out, the structure of the carbonaceous materials may be deformed, or the substrate may be damaged, and there may be problems wherein the device does not achieve desired performance or application thereof is limited.

Meanwhile, carbonaceous nanoparticle materials such as carbon nanotubes and graphene are more likely to be used as device materials for manufacturing biosensors since they have a superior ability to bind to phages or antibodies than metal nanoparticle materials. The present inventors have disclosed methods of manufacturing biosensors using a graphitic material, which is a carbonaceous nanomaterial, in Korean Patent No. 1694942, entitled “Biosensor and wearable device for detecting information of living bodies comprising hybrid electronic sheets” and Korean Patent Application No. 2016-0150329, entitled “enzyme film and biosensor having high sensitivity and specificity comprising the same”. In this disclosure, reported is a method of manufacturing a nanomaterial film by hydrodynamic immobilization via dialysis using a membrane. In this case, a colloidal material is also prepared by adding a graphitic material to a solution including a surfactant and stabilizing the same. Although an enzyme-integrated film may be realized by applying a hydrodynamic process to the entire structure of an enzyme and an electrode instead of using a conventional stacking method, there are problems in that the processing time in terms of days is long, and technical difficulties in mass production via automation of all or part of the manufacturing process.

### SUMMARY

One or more embodiments include a method of producing a nanoparticle device including: printing a colloidal composition on a hydrogel in a pattern, the colloidal composition including nanoparticles and a surfactant; and forming a nanoparticle device by removing the surfactant included in the colloidal composition through pores inside the hydrogel, and a nanoparticle device produced according to the production method.

Additional aspects will be set forth in part in the description which follows and, in part, will be apparent from the description, or may be learned by practice of the presented embodiments of the disclosure.

According to one or more embodiments, a method of producing a nanoparticle device includes: printing a colloidal composition including nanoparticles and a surfactant on a hydrogel in a pattern; and forming a nanoparticle device by removing the surfactant included in the colloidal composition through pores inside the hydrogel without damaging the printed pattern, and a nanoparticle device is produced according to the production method. According to one or more embodiments, a method of printing an electrode device includes the above-described operations.

In the printing of the colloidal composition on the hydrogel in the pattern, the colloidal composition may refer to an aqueous solution in which nanoparticles are dispersed or dissolved. The colloidal composition may be prepared by adding the nanoparticles to a solution including a surfactant and stabilizing the same due to a low dispersibility of the nanoparticles therein.

As used herein, the term “colloidal composition” refers to a composition directly printed, in a liquid phase, on a hydrogel to form a pattern and used in an inkjet printing process. Throughout the specification, the term “printing” may refer to printing using an inkjet printer, stencil printing, or the like. In an example embodiment, the colloidal composition may be mounted on an inkjet printer and printed on a hydrogel through a nozzle, or the like, of the printer. In an example embodiment, the colloidal composition including the nanoparticles and the surfactant is distinguished from that laminated on a substrate in the form of a sheet or film by a method such as lithography.

According to an example embodiment, the colloidal composition may be printed on the hydrogel, e.g., printed on the hydrogel by inkjet printing, transferred to the substrate, and dried, to form a device. Also, for example, the device may be a flexible electrode device, a transparent electrode device, a biosensor device, a strain sensor device, a pressure sensor device, a memory device, a logic device, an energy device, or an electrochemical device.

Also, the surfactant may include a bio-compatible surfactant compatible with a biomaterial such as a peptide or a phage. Examples of the surfactant may include sodium cholate (SC), sodium dodecyl sulfate (SDS), sodium deoxycholate (DOC), Nonidet P-40, Triton X-100, Tween 20®, polyethylene glycol (PEG) 600, sodium lauryl sulfate (SLS), ammonium-oleate, cetyltrimethyl ammonium bromide (CTAB), hydrolyzed tetraethyl orthosilicate (TEOS), or any mixture thereof.

As used herein, the term “nanoparticle” refers to a particle, at least one dimension of which is 100 nm or less, i.e., a particle having 1/ten million meters in size, belonging to nanotechnology used to manufacture a novel structure, material, machine, or device via manipulation of matter on an atomic and molecular scale and study a structure thereof. For example, the nanoparticles may be: graphitic materials such as graphene, highly oriented pyrolytic graphite (HOPG), graphene oxide, reduced graphene oxide, single-walled carbon nanotubes, double-walled carbon nanotubes, multi-walled carbon nanotubes, and fullerene; metallic nanoparticles such as metal nanowires, silver nanoparticles, platinum nanoparticles, gold nanoparticles, metal nanobeads, and magnetic nanoparticles; oxide nanoparticles such as silicon oxide, tungsten oxide, zinc oxide, neodymium oxide, titanium oxide, cerium oxide, and iron oxide; a nitride nanoparticle such as boron nitride and titanium nitride; a sulfide nanoparticle such as molybdenum disulfide (MoS₂) and tungsten disulfide (WS₂), or any mixture thereof. The metal of the metal nanobeads may be Au, Ag, Pt, Pd, Ir, Rh, Ru, Al, Cu, Te, Bi, Pb, Fe, Ce, Mo, Nb, W, Sb, Sn, V, Mn, Ni, Co, Zn, La, Ce, Y, Ti, Sc, Lu, Yb, Tm, Er, Ho, Dy, Tb, Gd, Eu, Sm, Pm, Nd, or Ce.

Meanwhile, as used herein, the term “graphitic material” refers to a material having a surface in which carbon atoms are arranged in a hexagon structure, i.e., a graphene surface, and any material having a graphitic surface may be regarded as the graphitic material regardless of physical, chemical, or structural properties. For example, the graphitic materials may include graphene sheets, HOPG sheets, carbon nanotubes such as single-walled carbon nanotubes, double-walled carbon nanotubes, and multi-walled carbon nanotubes, or fullerene. The graphitic materials may be metallic, semiconductor, or hybrid materials, e.g., a mixture of graphene sheets and single-walled carbon nanotubes.

As used herein, the term “hydrogel”, also referred to as hydrated gel, and having a network structure in which water-soluble polymers are three-dimensionally cross-linked via physical bonds (hydrogen bonds, van der Waals forces, hydrophobic interactions, or polymer crystals) or chemical bonds (covalent bonds), means a material that is not dissolved in an aqueous environment and contains a large amount of water due to pores formed therein. The hydrogel made from various water-soluble polymers may have various chemical compositions and physical properties. Also, the hydrogel may be easily processed and modified into various forms according to application. The hydrogel available in an example embodiment may be, for example, agarose gel, collagen, silicone, dextran, methylcellulose, hyaluronic acid, polyethylene oxide, polyvinyl pyrrolidone, polyvinyl alcohol, sodium polyacrylate, polyethylene glycol, acrylate polymer, acrylamide polymer, methacrylate polymer, or any mixture thereof. More particularly, the hydrogel may be 0.1 wt % to 50 wt %, 0.1 wt % to 10 wt %, 0.1 wt % to 5 wt %, 0.5 wt % to 20 wt %, 0.5 wt % to 10 wt %, 1 wt % to 10 wt %, 2 wt % to 30 wt %, 2 wt % to 10 wt %, or 2 wt % to 5 wt % agarose gel. A hydrogel available in another example embodiment may have a tensile strength, as a mechanical strength, of 20 kPa to 1000 kPa, 10 kPa to 800 kPa, 50 kPa to 300 kPa, or 100 kPa to 500 kPa.

In an example embodiment, the pores inside the hydrogel may have a diameter of 1 nm to 10 μm. More particularly, the diameter of the pores may be 1 nm to 10 μm, 10 nm to 10 μm, 100 nm to 10 μm, 500 nm to 10 μm, 1 μm to 10 μm, 200 nm to 5 μm, 500 nm to 5 μm, 200 nm to 1 μm, or 500 nm to 2 μm. That is, the diameter of the pore may be smaller than that of the nanoparticle and greater than a molecule of the surfactant. Thus, the surfactant may be diffused into water through the pores inside the hydrogel and the nanoparticles may form the nanoparticle device without damaging the printed pattern.

According to one or more embodiments, a composition for removing a surfactant dispersed in a nanoparticle aqueous solution, the composition including a hydrogel having pores with a diameter of 1 nm to 10 μm. The hydrogel included in the composition is used to prepare the nanoparticle device by removing the surfactant through the pores inside the hydrogel without damaging the pattern after printing the colloidal composition including the nanoparticle and the surfactant on the hydrogel in the pattern.

In an example embodiment, the colloidal composition may further include a peptide having the ability to bind to a carbonaceous material or a phage displaying a peptide having the ability to bind to a carbonaceous material. More particularly, the colloidal composition may include a solution containing the peptide or the phage displaying the peptide and a colloidal solution including the nanoparticle. In addition, the solution containing the peptide or the phage displaying the peptide may be a solution in which the peptide or the phage is dispersed. For example, the solution in which the phage is dispersed may include the phage at a concentration of 1×10¹⁰ number/ml to 1×10¹⁵ number/ml. The solution in which the peptide is dispersed may include the peptide at a concentration of 0.2 mg/ml to 4 mg/ml. Also, the colloidal solution may be a solution including carbon nanotubes (e.g., single-walled carbon nanotubes) at a concentration of 1×10¹⁰ number/ml to 1×10¹⁵ number/ml. The solution in which the peptide or the phage is dispersed may be mixed with the colloidal solution, in a volume ratio of, for example, 1:8 to 8:1. More particularly, the nanoparticle and the phage may be mixed in a molar ratio of 20:1 to 1:20.

By using the nanoparticles of the device according to an example embodiment, a binding affinity between the carbonaceous materials, as the nanoparticles, or a binding affinity between the nanoparticles and the substrate may be enhanced. More particularly, the peptide or the phage promotes binding between the nanoparticles, contributing to structurization/stabilization of the nanoparticles. This is a concept distinguished from functionalization of the graphitic material may indicate enabling formation of a network structure of the graphitic material. The nanoparticle may improve stability, particularly, in an aqueous solution, compared with a case in which the peptide or the phage displaying the peptide is not used.

Therefore, in an example embodiment, the peptide or the phage binding to the nanoparticle may serve as a binder composition or a bio-adhesive. Throughout the specification, the term “bio-adhesive” or “binder composition” may mean that the peptide or phage promoting binding between nanoparticles, e.g., the graphitic materials, or contributing adhesion properties between the graphitic material and the substrate. More particularly, because the phages displaying the peptide and having the ability to bind to the carbonaceous material specifically binds to the carbonaceous material, the phages may be introduced into interfaces between the elements of an energy device or an electrochemical device to improve adhesive properties or interfacial properties. For example, when the composition is introduced between a current collector and an active material of an energy device, the active material is adhered with a stronger force to the current collector, thereby increasing a thickness of the active material. Also, for example, when the composition is introduced into an active material of an energy device, interface separation of the active material may be prevented. Thus, the colloidal composition further including the peptide or the phage according to an example embodiment may improve adhesive properties between a current collector and an active material or between an active material and a separator of an electrochemical device or improve interfacial properties of the active material. Accordingly, the colloidal composition further including the peptide or the phage may be used for an electrode, a flexible device, an energy device, or an electrochemical device. Examples of the energy device may include a flexible battery, an alkaline battery, a dry battery, a mercury cell, a lithium battery, a nickel-cadmium battery, a nickel-hydrogen battery, and a secondary battery such as a lithium-ion secondary battery or a lithium-ion polymer secondary battery.

In another example embodiment, because the carbonaceous material or the graphitic material is not charged, additional functionalization is required to attach an enzyme for a biosensor or the like thereto. However, the peptide or the phage with the ability to bind to the carbonaceous material or the graphitic material is charged, an enzyme may be integrally bound into the network structure of the carbonaceous material or the graphitic material by electrolyte coating or the like. Thus, the phage or peptide may form junctions of a plurality of carbonaceous materials or graphitic materials. Also, the peptide may be formed of two peptides linked via a linker and link two carbonaceous materials or graphitic materials. More particularly, in a network structure of a plurality of graphitic materials, each of the two peptides linked via the linker may bind to each of the graphitic materials. The linker may be a peptide linker. The peptide linker may be one of various linkers known in the art, for example, a linker including a plurality of amino acids. According to an example embodiment, the linker may be a polypeptide including, e.g., 1 to 10 amino acids or 2 to 8 amino acids. The peptide linker may include Gly, Asn, and Ser residues, and may also include neutral amino acids such as Thr and Ala. Appropriate amino acid sequences for the peptide linker are well known in the art.

The peptide may be any peptide having the ability to bind to a graphitic material, e.g., a carbonaceous material. For example, the peptide according to the present disclosure may have an amino acid sequence selected from SEQ ID NOS: 1 to 12. The peptide may include a conservative substitution of the disclosed peptide. As used herein, the term “conservative substitution” refers to a substitution of a first amino acid residue with a second different amino acid residue. In this regard, the first and second amino acid residues refer to those having side chains with similar biophysical properties. The similar biophysical properties may include the ability to provide or accept hydrophobicity, charge, polarity, or hydrogen bond. Examples of the conservative substitution may include basic amino acids (arginine, lysine, and histidine), acidic amino acids (glutamic acid and aspartic acid), polar amino acids (glutamine and asparagine), hydrophobic amino acids (leucine, isoleucine, valine, and methionine), hydrophilic amino acids (aspartic acid, glutamic acid, asparagine, and glutamine), aromatic amino acid (phenylalanine, tryptophan, tyrosine, and histidine), and small amino acids (glycine, alanine, serine, and threonine). General amino acid substitutions that do not alter specific activity are known in the art. Thus, in the peptide, for example, X₁ may be W, Y, F, or H, X₂ may be D, E, N, or Q, and X₃ may be I, L, or V. Also, the peptide may be a peptide or a peptide set including at least one amino acid sequence selected from SEQ ID NOS: 5 to 11. Besides, the peptide may be a peptide that is not derived from the phage, e.g., a peptide having an amino acid sequence of SEQ ID NO: 12. The peptide may be a peptide formed of two peptides linked via a linker (e.g., SEQ ID NO: 10 or SEQ ID NO: 11). The peptide may further include a partial sequence of a coat protein of the phage, for example, 1 to 10 amino acid residues (e.g., SEQ ID NO: 9). A continuous amino acid sequence of the coat protein of the phage may be linked to an N-terminal or a C-terminal of an amino acid sequence of the peptide or peptide set. Thus, for example, the peptide or peptide set may have a sequence length of 5 to 60 amino acids, 7 to 55 amino acids, 7 to 40 amino acids, 7 to 30 amino acids, 7 to 20 amino acids, or 7 to 10 amino acids. The peptide or peptide set may be an assembled, (e.g., self-assembled) peptide or peptide set. For example, the peptide or peptide set may have an α-helix structure or a β-sheet structure. The peptide or peptide set may improve binding between the graphitic materials such that the graphitic materials have a mesh structure.

The peptide binding to the graphitic material may be selected from peptide libraries by a phage display method. By the phage display method, the peptide may be genetically linked to, is inserted into, or replaces the coat protein of the phage to be displayed on the surface of the phage and the peptide may be encoded by genetic information in a virion. The peptide may be selected by screening the displayed protein and various variants produced by DNA encoding the peptide, which is called “biopanning”. In summary, a biopanning technique includes reacting phages displaying various variants with an immobilized target (e.g., a graphitic material), washing unbound phages, and eluting phages specifically binding to the target by destroying binding interactions between the phages and the target. After a portion of the eluted phages may be left for DNA sequencing and peptide identification, the remainder may be amplified in vivo and a sub-library for the next round may be generated to repeat this process.

Also, the peptide may be displayed on the coat protein of the phage. Thus, for example, the phage having the ability to bind to the graphitic material may include the peptide displayed on the coat protein of the phage or a fragment thereof.

The terms “phage” and “bacteriophage” are used interchangeably and may refer to a virus that infects bacteria and replicates in bacteria. The phage or bacteriophage may be used to display a peptide selectively or specifically binding to a graphitic material or a volatile organic compound. The phage may be genetically engineered to display the peptide having the ability to bind to the graphitic material on the coat protein of the phage or a fragment thereof. As used herein, the term “genetic engineering” or “genetically engineered” refers to introducing at least one genetic modification into the phage to display the peptide having the ability to bind to the graphitic material on the coat protein of the phage or a fragment thereof, or a phage manufactured thereby. The genetic modification includes introducing a foreign gene encoding the peptide. In addition, the phage may be filamentous phage, e.g., M13 phage, F1 phage, Fd phage, If1 phage, Ike phage, Zj/Z phage, Ff phage, Xf phage, Pf1 phage, or Pf3 phage.

As used herein, the term “phage display” refers to displaying a functional foreign peptide or a protein on the surface of phage or phagemid particles. The surface of the phage may refer to the coat protein of the phage or a fragment thereof. In addition, the phage may be a phage prepared by linking the C-terminal of the functional foreign peptide to the N-terminal of the coat protein of the phage, inserting the peptide into a continuous amino acid sequence of the coat protein of the phage, or substituting a portion of the continuous amino acid sequence of the coat protein with the peptide. The insertion or substitution of the peptide may take place at an amino acid position of 1^{st} to 50^{th}, 1^{st} to 40^{th}, 1^{st} to 30^{th}, 1^{st} to 20^{th}, 1^{st} to 10^{th}, 2^{nd} to 8^{th}, 2^{nd} to 4^{th}, 2^{nd} to 3^{rd}, 3^{rd} to 4^{th}, or 2^{nd} from the N-terminal of the coat protein in the amino acid sequence of the coat protein. Also, the coat protein may be p3, p6, p8, or p9. In an example embodiment, the phage may be M13 phage genetically engineered to have the ability to bind to the nanoparticle, the M13 phage may be a phage displaying a peptide having at least one amino acid sequence selected from SEQ ID NO: 1 and SEQ ID NO: 9, and the peptide may be displayed on the coat protein P3, P6, P7, P8 or P9 of the M13 phage.

In another example embodiment, the graphitic material may have a network structure, and the enzyme may be located on the network structure, in the network structure, and/or below the network structure. In addition, the network structure may be formed by a bound complex of the graphitic material and the peptide or a bound complex of the graphitic material and the phage. Thus, the internal structure of the graphitic material may be a percolated network structure. As used herein, the term “percolated network” may refer to a lattice structure formed of random conductive or non-conductive connections.

In the printing of the colloidal composition on the hydrogel in the pattern, the pattern may be any pattern designed for a desired purpose, e.g., a pattern for an electrode including a line pattern, a pattern for a strain sensor, a pattern for a pressure sensor, a pattern for a biosensor, a pattern for a memory, a pattern for a logic device, a pattern for an energy device, a pattern for an electrochemical device, a film, a sheet, or a 3D pattern. As used herein, the term “3D pattern” refers to the pattern for an electrode, the pattern for a strain sensor, the pattern for a pressure sensor, the pattern for a biosensor, the pattern for a memory, the pattern for a logic device, the pattern for an energy device, the pattern for an electrochemical device, the film, the sheet, or the like formed on the surface of or inside a 3D structure. In an example embodiment, formation of the 3D pattern may be freely adjusted according to an easily adjustable shape of the hydrogel. It is easy to form a pattern on a 3D surface due to characteristics of the colloidal composition and the printing method.

In addition, the printing of the colloidal composition in the pattern may be performed by repeating the printing twice or 20 times, i.e., multi-stacking the same colloidal composition or different colloidal compositions. The printing may be performed in different directions, the colloidal compositions of the respective layers may be different, and the patterns of the respective layers may also be different. By changing the direction of printing, performance of the device may vary. More particularly, a multilayered structure may be formed by repeating the printing twice to 10 times, 4 times to 10 times, or 4 times to 6 times. The repeating of the printing to a multilayered structure is referred to as “multi-stacking” throughout the specification. The multi-stacking enables molding in various shapes and stacking a 3D patterns as well as films. In addition, when an order of the layers is important as in the case of a biosensor, utilization thereof may increase.

According to one or more embodiments, the removing of the surfactant included in the colloidal composition through pores inside the hydrogel without damaging the printed pattern may further include contacting the hydrogel with a liquid for faster removal thereof. More particularly, the hydrogel may be immersed in a liquid, e.g., distilled water, to the extend lower than a height of the hydrogel, for 10 minutes to 5 hours, 10 minutes to 2 hours, 20 minutes to 2 hours, 30 minutes to 2 hours, 30 minutes to 1 hour, or 20 minutes to 1 hour. Accordingly, the surfactant included in the composition to improve dispersity may be removed without damaging the pattern.

According to one or more embodiments, the method of producing the nanoparticle device may further include transferring the nanoparticle device formed on the hydrogel to a substrate. The transferring of the nanoparticle device to the substrate may be performed by contacting the substrate with an upper surface of the hydrogel. The transferring is performed based on the principle of difference in interfacial energy between the hydrogel and the substrate. As used herein, the term “contact transfer” refers to a method of transferring via direct contact with the substrate as described above. According to a contact transfer method, the pattern formed on the hydrogel may become upside-down. In the case of a multi-stacked structure according to an example embodiment, an uppermost layer will be a lowermost layer in a final device. In an example embodiment, the method may further include drying the transferred composition. In another example embodiment, the method may further include immobilizing an enzyme on the dried composition. The enzyme may be an analyte binding material. As used herein, the terms “analyte binding material” and “analyte binding reagent” are used interchangeably and may refer to a material specifically binding to an analyte. The analyte binding material may include a redox enzyme. The redox enzyme may refer to an enzyme that oxidizes or reduces a substrate and may include, for example, oxidase, peroxidase, reductase, catalase, or dehydrogenase. Examples of the redox enzyme may include blood glucose oxidase, lactate oxidase, cholesterol oxidase, glutamate oxidase, horseradish peroxidase (HRP), alcohol oxidase, glucose oxidase (GOx), glucose dehydrogenase (GDH), cholesterol esterase, ascorbic acid oxidase, alcohol dehydrogenase, laccase, tyrosinase, galactose oxidase, or bilirubin oxidase. The enzyme may be included in the graphitic material in a state of being immobilized on or in the mesh structure. The term “immobilized” may refer to a chemical or physical bond between an enzyme and a graphitic material. The method may further include treating a polymeric material on the formed device. The composition according to an example embodiment may be reformed with a positively charged polymer or a negatively charged polymer, although there is no need for a separate reforming process for the composition. For example, the positively charged polymer may be poly(allyamine) (PAH), polydiallyldimethylammonium (PDDA), polyethylenei mine ethoxylated (PEIE), or poly(acrylamide-co-diallyldimethylammonium) (PAMPDDA). Also, the negatively charged polymer may be, for example, poly(4-styrenesulfonate) (PSS), poly(acrylic acid) (PAA), poly(acryl amide) (PAM), poly(vinylphosphonic acid), poly(2-acrylamido-2-methyl-1-propanesulfonic acid) (PAAMP), poly(anetholesulfonic acid) (PATS), or poly(vinyl sulfate) (PVS).

The substrate may be a conductive or insulating substrate. A material of the substrate may include a metal, a semiconductor, an insulator, a polymer, an elastomer, or the like. For example, the substrate may be a quartz substrate or a gold substrate. According to an embodiment, the substrate may be a transparent flexible substrate. For example, the transparent flexible substrate may be a substrate formed of polydimethylsiloxane (PDMS), polyethersulfone (PES), poly(3,4-ethylenedioxythiophene), poly(styrenesulfonate), polyimide, polyurethane, polyester, perfluoropolyether (PFPE), polycarbonate, or any combination of the polymers. In an example embodiment, a flexible electronic device may be prepared by detaching the nanoparticle pattern using a flexible polymer substrate and drying the nanoparticle pattern. In an example embodiment, the nanoparticle pattern may be transferred to a flexible stretchable substrate such as a polymer glove for experiments. The pattern designed according to an example embodiment may be printed on the hydrogel, transferred to the quartz substrate, and dried to form an electrode, e.g., an electrode device, more particularly, a transparent electrode.

Thus, according to one or more embodiments, a transparent electrode includes the device produced according to the production method of the present disclosure. Although metals have been line-patterned due to low conductivity of a transparent electrode, carbon nanotubes cannot be line-patterned by existing methods in which a surfactant is removed by washing/acid-treatment. However, a transparent electrode may be manufactured using carbon nanotubes by enabling line patterning using the production method according to the present disclosure.

According to one or more embodiments, the transferring of the nanoparticle device to the substrate may include: separating the nanoparticle device from the hydrogel by adding a liquid in which the nanoparticle device formed on the hydrogel is able to float; and transferring the nanoparticle device to the substrate by contacting the substrate with a surface of the floating nanoparticle device facing the hydrogel. Throughout the specification, such a transferring method is referred to as “floating transfer method”. The pattern prepared according to an example embodiment may be printed on the hydrogel in a multilayered structure, floating on water, and transferred to the substrate to form an electrode. For example, a biosensor electrode may be prepared by printing an ink including a carbonaceous material and a biomaterial, printing polyethyleneimine (PEI) thereon to form an intermediate layer, and printing a GOx enzyme thereon. The prepared biosensor electrode is transferred to a commercial electrode by floating the biosensor electrode on water, and thus an electrode may be configured to satisfy requirements for a device, in which the order of printing and the surface layer are important, and the upper and lower layers of the biosensor electrode may not be inverted. Thus, according to the method of producing a nanoparticle device according to the present disclosure, a multi-layer is formed by stacking another functional layer onto the composition or immobilizing an enzyme onto the composition, and the multi-layer is transferred to the substrate with the stacking order maintained. In addition, in the case of forming an electrode on a substrate having a nanometer or micrometer scale pattern, it may be difficult to form a desired shape due to the shape of the pattern and characteristics of inkjet printing. When a desired pattern is printed on the hydrogel and transferred by floating, the electrode may be formed and transferred without being affected by the shape of the substrate, and thus a high-performance device may be prepared.

According to one or more embodiments, a biosensor includes the nanoparticle device produced according to the production method. The biosensor may further include a test cell for accommodating a sample, a substrate, and an enzyme electrode, and the test cell may include a channel having an inlet or an outlet for introducing or discharging the sample.

The biosensor according to an example embodiment may include a test cell including a channel formed on a substrate with a working electrode (WE), a counter electrode (CE), and a reference electrode (RE) located thereon. The test cell has an inlet for introducing a sample or an outlet for discharging the sample. The sample may be introduced through the inlet and the analyte included in the sample may participate in a redox-reaction with an enzyme to cause an electrochemical potential gradient. The term “chemical potential gradient” may refer to a concentration gradient of a redox-active material. When the gradient is present between two electrodes, a potential difference may be detectable when a circuit is opened, and when the circuit is closed, a current may flow until the gradient is reduced to zero. The chemical potential gradient may mean a gradient of potential formed by a potential different or current flow between the electrodes generated by asymmetric distribution of the redox enzyme (e.g., analyte binding material). In a biosensor according to an example embodiment, a strong peak of the redox reaction is observed in a working electrode on which the enzyme electrode is transferred, and the redox peak slightly occurs or does not occur in the other electrodes. Thus, in the biosensor according to an example embodiment, the migration of electrons due to the redox reaction between an analyte and an enzyme may be direct electron transfer (DET) on a working electrode with an enzyme electrode transferred thereon in the absence of a medium.

As used herein, the term “analyte” may refer to a material of interest that may be present in a sample. A detectable analyte may include materials involved in a specific-binding interaction with one or more analyte-binding materials, which participate in a sandwich, competitive, or replacement assay configuration. Examples of the sample may include blood, body fluid, cerebrospinal fluid, urine, excreta, saliva, tear, or sweat. Examples of the analyte may include antigens such as peptides (e.g., hormone) or heptens, proteins (e.g., enzyme), carbohydrates, proteins, drugs, agricultural chemicals, microorganisms, antibodies, and nucleic acids participating in sequence-specific hybridization with complementary sequences. More particular examples of the analyte may include glucose, cholesterol, lactate, hydrogen peroxide, catechol, tyrosine, and galactose.

A biosensor according to an example embodiment may further include a meter for determination of an analyte. As used herein, the term “determination of an analyte” refers to qualitative, semi-quantitative and quantitative processes for evaluating a sample. In a qualitative evaluation, a result indicates whether or not the analyte is detected in the sample. In a semi-quantitative evaluation, the result indicates whether or not the analyte is present above some predefined threshold. In a quantitative evaluation, the result is a numerical indication of the amount of the analyte present.

The meter may include an electronic device that measures a potential difference or current at a predetermined time after a sample is introduced, and converts a measurement value into a numerical indication. The measuring of the potential difference or current may be determining of an oxidation current reaction voltage value by using cyclic voltammetry (CV). According to the CV, a potential of a first electrode (e.g., a working electrode) is circulated at a predetermined rate to measure a current. The converting of the measurement value may be performed by referring to a look-up table that is used to convert a value of a current or potential into a value of an analyte dependent on a specific device structure and a correction value with respect to the analyte. Also, the meter may further include a display showing results and a frame including at least one controlling interface (for example, a power button or a scroll wheel). The frame may include a slot for receiving a biosensor. The frame may include a circuit inside to apply a potential or current to an electrode of the biosensor when a sample is provided. A suitable circuit for the meter may be a suitable voltage meter that measures a potential crossing the electrode. Also, provided is a switch that is opened when the potential is measured or is closed when the current is measured. The switch may be a mechanical switch (for example, relay), or a field-effect transistor (FET) switch, or a solid-state switch. The circuit may be used to measure a potential difference or a current difference. As understandable to one of ordinary skill in the art, other circuits including more simple or complicated circuits may be used to apply a potential difference, a current, or both.

According to one or more embodiments, a wearable device includes the biosensor. The wearable device may be used for detecting bioinformation. The wearable device may be a patch, a watch, or a contact lens. The biosensor may exhibit remarkable electrochemical properties on a transparent flexible substrate and an electrode harmless to the human body. Further, the biosensor does not need a mediator harmful to the human body and has high sensitivity enough to detect a small amount of an analyte in a sample.

According to one or more embodiments, a nanoparticle device is produced according to the production method of the present disclosure. The nanoparticle device prepared according to the production method according to the present disclosure has a high degree of homogeneity of the nanoparticles over the entire pattern due to excellent dispersibility, has a high binding affinity between nanoparticles by removing the surfactant, and has excellent electrical activity due to high accuracy of the pattern. Thus, the nanoparticle device may be applied to implementation of 3D pattern which could not be realized by conventional methods, implementation of a nanoparticle device on a polymer glove, or the like, and line pattering of nanoparticles such as carbon nanotubes. Also, because dialysis is performed in a liquid, freestanding is required and thus addition of a biomaterial such as a phage is required according to conventional membrane dialysis methods. However, the device according to the present disclosure is different therefrom because addition of a biomaterial such as a phage is optional. Also, because washing or heat/acid treatment is not required to remove the surfactant, a device may be provided without damaging a pattern.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a flowchart of a method of preparing a nanoparticle device according to an embodiment;

FIG. 2 is a diagram illustrating a process of removing a surfactant (102) included in a colloidal composition through pores (103) of a hydrogel (104) after the colloidal composition is printed onto the hydrogel (104) in a flat pattern (101);

FIG. 3 is a diagram illustrating a nanoparticle device prepared by printing a complex pattern (101) onto a hydrogel (104);

FIG. 4 is a diagram illustrating a 3D pattern formed by uniformly printing a pattern (101) onto a 3D hydrogel (104);

FIG. 5 is a flowchart of a method of producing a nanoparticle device according to the present disclosure, including transferring the nanoparticle device to a substrate by contacting the substrate with an upper surface of a hydrogel;

FIG. 6 is a diagram illustrating a nanoparticle device prepared by printing a double-layered pattern (101, 106) onto a hydrogel (104) and transferring the pattern to a final substrate (105) while upside-down by contacting the substrate (105) with an upper surface of the hydrogel;

FIG. 7 is a flowchart of a method of producing a nanoparticle device according to the present disclosure, including molding transfer by which a solution to be hardened is poured onto a hydrogel and the hardened solution is detached therefrom;

FIG. 8 is a flowchart of a method of producing a nanoparticle device according to the present disclosure, including multi-stacking;

FIG. 9 is a diagram illustrating a nanoparticle device produced in a triple layered structure (101, 106, 107) by repeating printing three times;

FIG. 10 is a flowchart of a method of producing a nanoparticle device according to the present disclosure, including floating transferring;

FIG. 11 is a diagram illustrating a floating transfer method by which a pattern is transferred from a hydrogel (104) to a final substrate (105) with a stacking order maintained;

FIG. 12 shows an image of an agarose-based hydrogel prepared according to an embodiment.

FIG. 13 shows an image of an acrylamide-based hydrogel prepared according to an embodiment.

FIG. 14 shows images of ejected ink solutions for printing, which are colloidal compositions (Preparation Examples 3 to 5);

FIG. 15 shows images of microstructures in which mono-layered, double-layered, and triple-layered circular patterns are formed by printing an ink solution (Preparation Example 3), which is a colloidal composition, onto an agarose-based hydrogel according to an embodiment, observed through an optical microscope;

FIG. 16 shows an image of a microstructure in which a penta-layered line pattern is formed by printing an ink solution (Preparation Example 3), which is a colloidal composition, onto a hydrogel according to another embodiment, observed through an optical microscope;

FIG. 17 shows an exemplary image of a triple-layered circular pattern formed by printing an ink solution (Preparation Example 3), which is a colloidal composition, onto an acrylamide-based hydrogel according to an embodiment;

FIG. 18 shows images of square mono-layered, double-layered, and triple-layered square patterns formed by printing an ink solution (Preparation Example 5), which is a colloidal composition, onto an agarose-based hydrogel, and transferred to a quartz substrate according to another embodiment;

FIG. 19 shows an image of a penta-layered line pattern formed by printing an ink solution (Preparation Example 4), which is a colloidal composition, onto an agarose-based hydrogel, and contact transferred to a PET substrate according to another embodiment;

FIG. 20 shows images of a triple-layered circular pattern formed by printing an ink solution (Preparation Example 3), which is a colloidal composition, onto an acrylamide-based hydrogel, and transferred to a quartz substrate according to an embodiment;

FIG. 21 shows images of a triple-layered circular pattern formed by printing an ink solution (Preparation Example 3), which is a colloidal composition, onto an acrylamide-based hydrogel, and molding transferred by pouring a PDMS solution and hardening the solution according to an embodiment;

FIG. 22 shows images a mono-layered square pattern formed by printing an ink solution (Preparation Example 3), which is a colloidal composition, onto a hydrogel, and immersed in and floated in water for transfer thereof according to an embodiment;

FIG. 23 shows images of a triple-layered circular pattern formed by printing an ink solution (Preparation Example 4) according to an embodiment, which is a colloidal composition, onto a hydrogel, and stamp transferred to a commercial electrode (Dropsense, BT250) by directly contacting a final substrate with the pattern;

FIG. 24 shows an image of an enzyme electrode formed by printing a colloidal composition, a polymer electrolyte, and an enzyme onto an agarose-based hydrogel, and floating transferred to a commercial electrode;

FIG. 25 is a graph illustrating sheet resistance of a transparent electrode prepared by printing an ink solution (Preparation Example 5), which is a colloidal composition, onto a hydrogel in a penta-layered or deca-layered structure and contact transferring the printed pattern to a quartz substrate according to an embodiment;

FIG. 26 is a graph illustrating transmittance of a transparent electrode prepared by printing an ink solution (Preparation Example 5), which is a colloidal composition, onto a hydrogel, in a penta-layered or deca-layered structure and stamp transferring the printed pattern to a quartz substrate according to an embodiment;

FIG. 27 is a graph illustrating direct-electron-transfer (DET) peaks of a glucose sensor (Preparation Example 11) including a nanoparticle device according to an embodiment;

FIG. 28 is a graph illustrating redox curves of a glucose sensor (Preparation Example 11) including a nanoparticle device according to an embodiment, with respect to glucose concentration;

FIG. 29 is a graph illustrating changes of reduction current peaks of a glucose sensor (Preparation Example 11) including a nanoparticle device according to an embodiment, with respect to glucose concentration;

FIG. 30 is a graph illustrating real-time monitoring characteristics of a glucose sensor (Preparation Example 11) according to an embodiment, and selectivity for uric acid;

FIG. 31 is a graph illustrating sensitivity of real-time monitoring characteristics of a glucose sensor (Preparation Example 11) according to an embodiment;

FIG. 32 is a graph illustrating redox curves of an all-printed enzyme electrode (Preparation Example 12) according to an embodiment, with respect to glucose concentration;

FIG. 33 is a graph illustrating changes of reduction current peaks of an all-printed enzyme electrode (Preparation Example 12) according to an embodiment, with respect to glucose concentration;

FIG. 34 is a graph illustrating selectivity for glucose of an all-printed enzyme electrode (Preparation Example 12) according to an embodiment;

FIG. 35 shows an image of a serpentine pattern formed by printing an ink composition (Preparation Example 3), which is a colloidal composition, onto an agarose-based hydrogel, and contact transferred to a polymer glove to produce a strain sensor device according to an embodiment; and

FIG. 36 is a graph illustrating changes of resistance of a nanoparticle device formed on a polymer glove according to an embodiment, with respect to various hand motions.

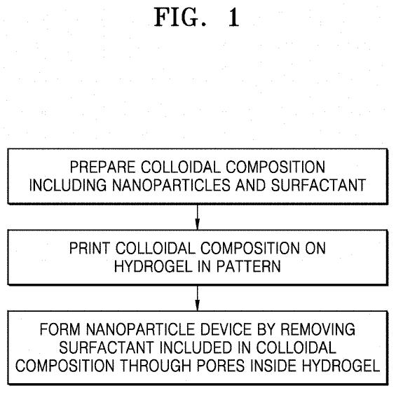

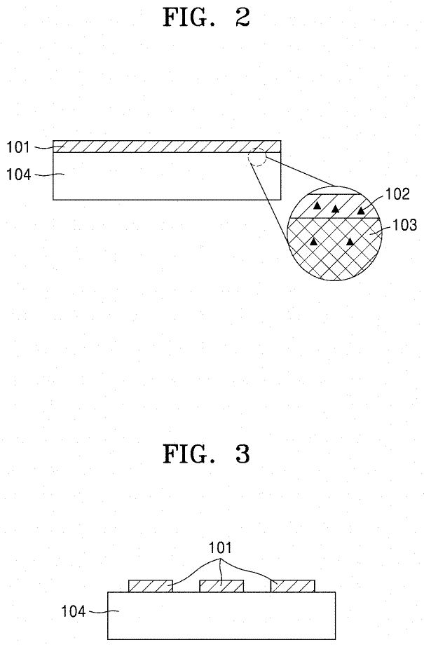

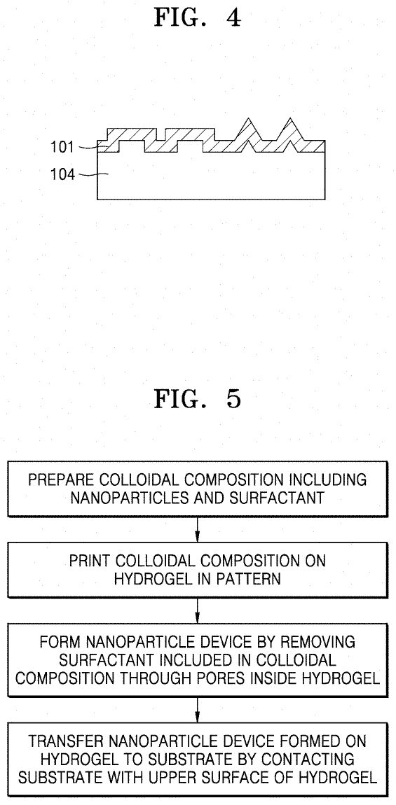

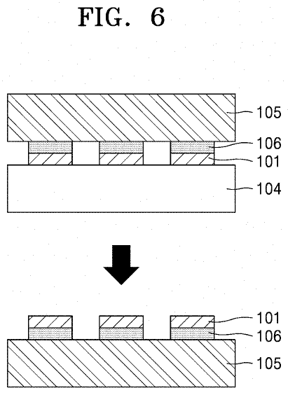

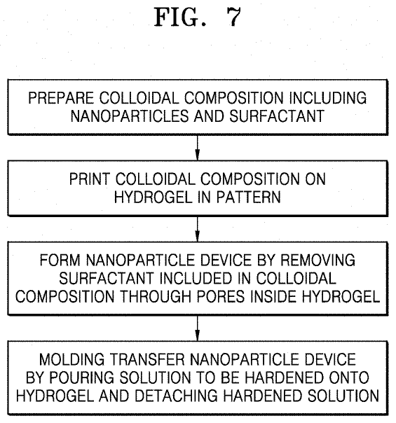

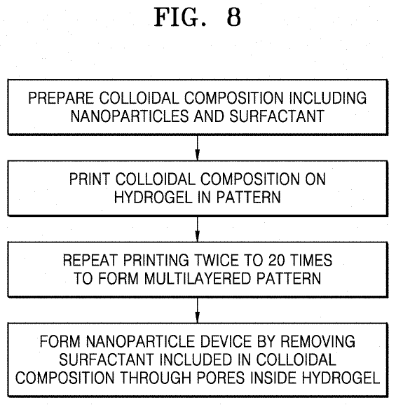

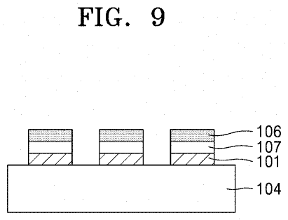

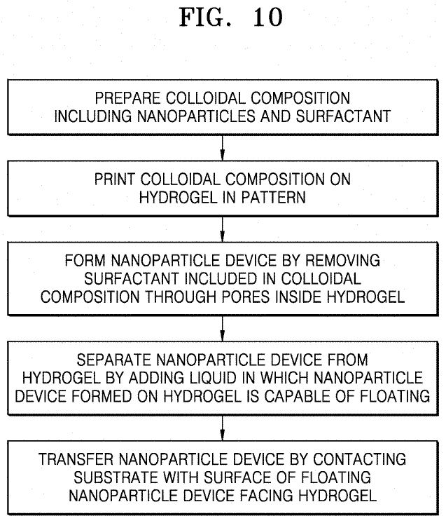

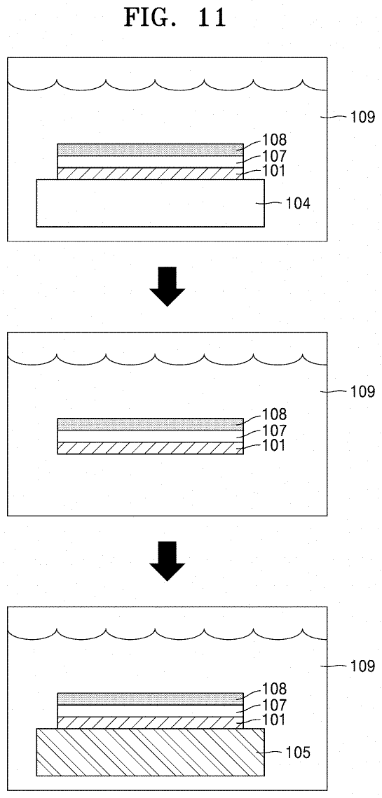

### DETAILED DESCRIPTION

A method of producing a nanoparticle device according to the present disclosure may be economical by using a hydrogel, may be easy to design a mass production process, and may reduce manufacturing time to 1/100 to 1/10 of conventional technology. In addition, a nanoparticle device may be produced in various designs by stably realizing a 3D pattern and stacking a pattern and may have excellent uniformity of nanoparticles and excellent electrical activity by removing a surfactant without damaging the pattern.

The nanoparticle device according to the present disclosure may have excellent homogeneity, high binding affinity and excellent electrical activity due to accuracy of a pattern and may be applied to stacking of pattern which could not be implemented by conventional methods.

Hereinafter, the present disclosure will be described in more detail with reference to the following examples. However, these examples are for illustrative purposes only, and the present disclosure is not intended to be limited by these examples.

### PREPARATION EXAMPLES

### Preparation Example 1_Preparation of Agarose-based Hydrogel

According to an embodiment of the present disclosure, an agarose-based hydrogel was prepared according to the following method.

A homogenous agarose aqueous solution was prepared at a concentration of 0.8 wt % to 20 wt % using a microwave oven. The solution was cooled to a temperature of 60° C. or less by storing the solution at room temperature, and then poured into a mold and maintained at room temperature for 3 hours or more for hardening. The hydrogel prepared in this manner is shown in FIG. 12.

### Preparation Example 2_Preparation of Acrylamide-based Hydrogel

According to an embodiment of the present disclosure, an acrylamide-based hydrogel was prepared according to the following method.

As a raw material, an acrylamide/bisacrylamide aqueous solution was prepared at a ratio of 19:1 to 37.5:1. Water was added thereto to prepare an aqueous solution at a concentration of 0.1% to 25% based on acrylamide. 10% ammonium persulfate solution was prepared as a hardener. After mixing the acrylamide/bisacrylamide aqueous solution and the ammonium persulfate solution, both prepared as described above, tetramethylethylenediamine, as a catalyst, was added to the mixture and mixed. The mixture was poured into a mold and maintained at room temperature for 30 minutes or more for hardening. The acrylamide-based hydrogel prepared in this manner is shown in FIG. 13.

### Preparation Example 3 Preparation of Ink Solution Including Carbonaceous Material

First, an aqueous solution was prepared by adding sodium-cholate, as a surfactant, to distilled water at a concentration of 1% or 2% w/v, and a colloidal solution was prepared by stabilizing single-walled carbon nanotubes (manufacturer: Nanointegris, SuperPure SWNTs, solution, concentration: 250 μg/mL or 1000 μg/mL), as a graphitic material, with the sodium-cholate by dialysis of the single-walled carbon nanotubes for 48 hours.

In this regard, assuming that an average length and an average diameter of the carbon nanotubes (CNTs) were 1 μm and 1.4 nm, respectively, the number of the single-walled carbon nanotubes in the colloidal solution is calculated according to the following equation.

$$Number of single \text{-} walled carbon nanotubes ( \frac{Number}{mL} ) = concentration ( \frac{µg}{mL} ) \times 3 \times {10}^{14} ( \frac{Number}{µg} ) \qquad Equation 1$$

According to Equation 1, the number of the single-walled carbon nanotubes included in 1000 μg/ml of the colloidal solution was 3×10¹⁴ CNT/mL. The number of the single-walled carbon nanotubes per unit volume was adjusted using the sodium-cholate aqueous solution having the same concentration as that of the dialyzed solution.

### Preparation Example 4 Preparation of Ink Solution Including Carbonaceous Material and Biomaterial

4-1. Preparation of Biomaterial

As M13 phages having a strong binding affinity to the graphitic surface, a M13phage (GP1) displaying a peptide SWAADIP (SEQ ID NO: 7) having a strong binding affinity to the graphitic surface and a phage (GP2) displaying NPIQAVP (SEQ ID NO: 8) were prepared according to the following method.

First, an M13HK vector was prepared by site-directed mutation of the 1381^{st} base pair C of an M13KE vector (NEB, product # N0316S, SEQ ID NO: 13) to G.

Here, the prepared M13KE vector (NEB, product # N0316S) was a cloning vector consisting of a 7222 bp DNA (Cloning vector M13KE), and genetic information thereof is available from the Internet (https://www.neb.com/˜/media/NebUs/Page %20Images/Tools %20and %20Resource s/Interactive %20Tools/DNA %20Sequences %20and %20Maps/Text %20Documents/m13kegbk.txt). Base sequences of oligonucleotides used for the site-directed mutation are as follows:

(SEQ ID NO: 14)
5′-AAG GCC GCT TTT GCG GGA TCC TCA CCC TCA GCA GCG
AAA GA-3′,
and
(SEQ ID NO: 15)
5′-TCT TTC GCT GCT GAG GGT GAG GAT CCC GCA AAA GCG
GCC TT-3′.

Phage display p8 peptide libraries were prepared from the prepared M13HK vector using restriction enzymes BspHI (NEB, product # R0517S) and BamHl (NEB, product # R3136T).

The base sequences of oligonucleotides used for the preparation of the phage display p8 peptide libraries are as follows:

(SEQ ID NO: 16)
5′-TTA ATG GAA ACT TCC TCA TGA AAA AGT CTT TAG TCC
TCA AAG CCT CTG TAG CCG TTG CTA CCC TCG TTC CGA
TGC TGT CTT TCG CTG CTG-3′,
and
(SEQ ID NO: 17)
5′-AAG GCC GCT TTT GCG GGA TCC NNM NNM NNM NNM NNM
NNM NNM NCA GCA GCG AAA GAC AGC ATC GGA ACG AGG
GTA GCA ACG GCT ACA GAG GCT TT-3′.

The nucleotide sequences of the prepared phage display p8 peptide libraries have a diversity of 4.8×10⁷ plaque-forming units (PFU) and each sequence has a copy number of about 1.3×10⁵. Then, the prepared phage display p8 peptide libraries were bound to a graphitic surface by biopanning to screen a phage displaying a peptide to be used as the biomaterial according to the present disclosure. The biopanning was conducted as follows.

First, a fresh surface of a highly oriented pyrolytic graphite (HOPG, SPI, product #439HP-AB) that has a graphitic surface was obtained before an experiment by attaching a tape thereto and detaching the tape therefrom to minimize defects caused by oxidation of a sample surface. In this regard, a HOPG substrate with a relatively large grain size of 100 μm or smaller was used.

Then, the prepared 4.8×10¹¹ (4.8×10⁷ diversities, 1000 copies per each sequence) phage display p8 peptide libraries were prepared in 100 μL of Tris-buffered saline (TBS) and conjugated with the HOPG surface in a shaking incubator for 1 hour at 100 rpm. 1 hour later, the solution was removed and the HOPG surface was washed 10 times with TBS. The washed HOPG surface was reacted with pH 2.2 Tris-HCl as an acidic buffer for 8 minutes to elute non-selectively reacting peptides and then XL-1 blue E. coli culture in mid-log state was eluted for 30 minutes. A portion of the eluted culture was left for DNA sequencing and peptide identification and the remainder was amplified to prepare sub-libraries for the next round. The above procedure was repeated using the prepared sub-libraries. Meanwhile, the left plaques were subjected to DNA analysis to identify the p8 peptide sequence. As a result, the phage (GP1) displaying the peptide SWAADIP (SEQ ID NO: 7) and the phage (GP2) displaying the peptide NPIQAVP(SEQ ID NO: 8), wherein the peptides have strong ability to bind to a graphitic surface, were obtained.

4-2. Preparation of Ink Solution Including Biomaterial

M13 phage (GP1) having a strong binding affinity to the surface of the graphitic material was dispersed in Tris-Buffered saline (TBS) at a concentration of 6×10¹³ number/mL. The colloidal solution prepared in Preparation Example 2 was mixed with the M13 phage solution at a volume ratio of 2:1 to mix the graphitic material with the M13 phage (GP1) at a molar ratio of 10:1. In this regard, the number of M13 particles included in the M13 phage solution was calculated according to the following equation. A_{269 nm} and A_{320 nm} indicate absorbances of the solution at wavelength of 269 nm and 320 nm, respectively.

$$Number of M 13 phage ( number \text{/} mL ) = \frac{\begin{matrix}\begin{matrix}{A}_{269 nm} - \\ {A}_{320 nm} \times \end{matrix} \\ 6 \times {10}^{17}\end{matrix}}{7234} \qquad Equation 2$$

### Preparation Example 5 Preparation of Ink Solution Including Carbonaceous Material with High Aspect Ratio

Single-walled carbon nanotubes having an average length of 15 μm were dispersed in a 2 wt % aqueous solution of sodium-cholate using a homogenizer. The solution was dispersed by a tip sonicator at a power of 1% for 15 minutes. The solution was centrifuged using a centrifuge at a relative centrifugal force of 90,000 g for 15 minutes, and a supernatant was extracted therefrom to prepare an ink solution including a carbonaceous nanomaterial with a high aspect ratio.

### Preparation Example 6 Preparation of Pattern on Agarose-based Hydrogel by Printing Ink Solution Including Carbonaceous Material and Ink Solution Including Carbonaceous Material and Biomaterial

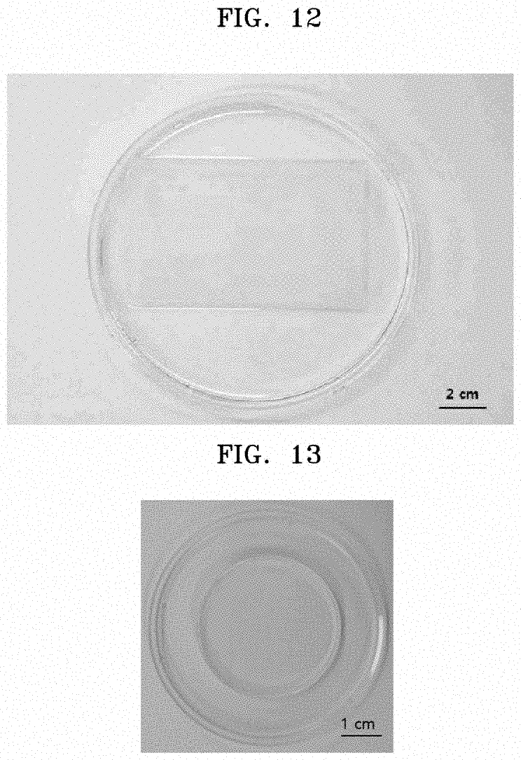

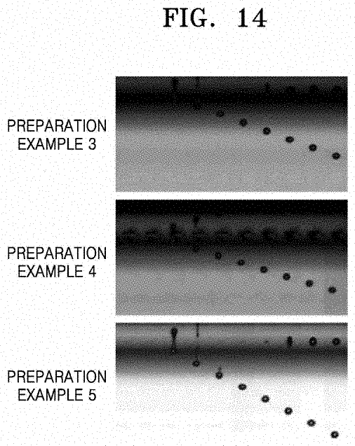

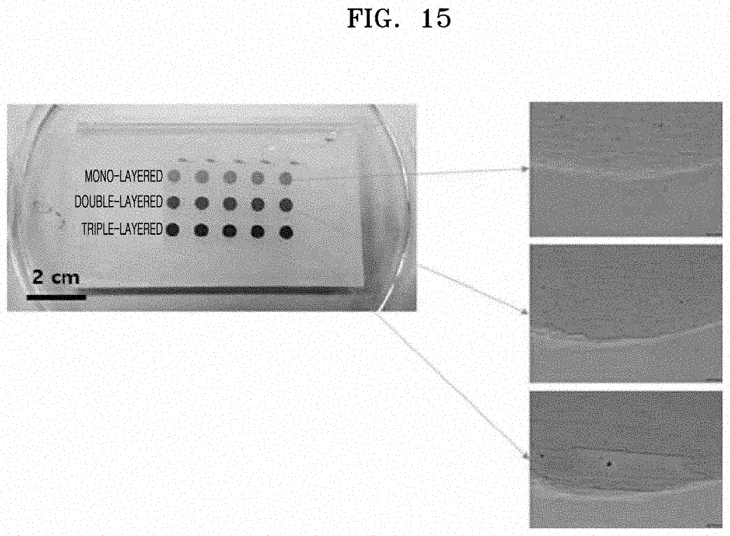

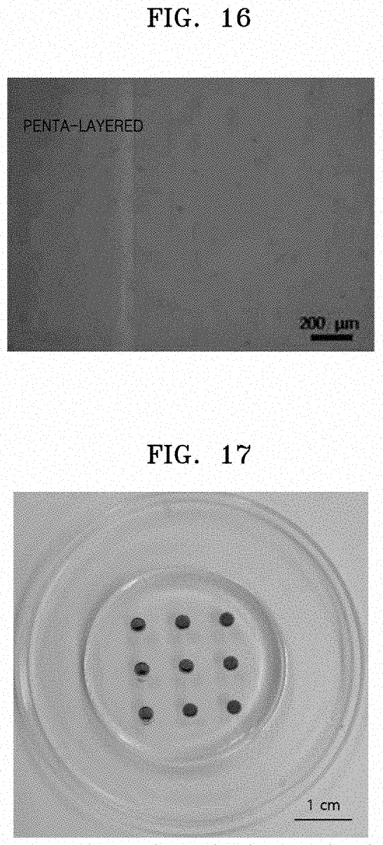

Each of the ink solutions prepared according to Preparation Examples 3 to 5 was printed on the hydrogel prepared in Preparation Example 1 in multiple layers to prepare an electrode for analysis of characteristics. Images of ejecting solutions are shown in FIG. 14. Subsequently, the hydrogel used as a substrate was immersed in water for 30 minutes or more to remove a surfactant and other materials used to prepare the ink solution, thereby preparing a pattern for transfer. A circular pattern prepared using the ink solution of Preparation Example 3 is shown in FIG. 15. A linear pattern prepared using the ink solution of Preparation Example 3 is shown in FIG. 16.

### Preparation Example 7 Preparation of Pattern on Acrylamide-based Hydrogel by Printing Ink Solution Including Carbonaceous Material and Ink Solution Including Carbonaceous Material and Biomaterial

Each of the ink solutions prepared according to Preparation Examples 3 to 5 was printed on the hydrogel prepared in Preparation Example 2 in multiple layers to prepare an electrode for analysis of characteristics. Subsequently, the hydrogel used as a substrate was immersed in water for 30 minutes or more to remove a surfactant and other materials used to prepare the ink solution, thereby preparing a pattern for transfer. A circular pattern prepared using the ink solution of Preparation Example 3 is shown in FIG. 17.

### Preparation Example 8 Preparation of Device by Contact Transfer of Pattern Prepared by Printing Process

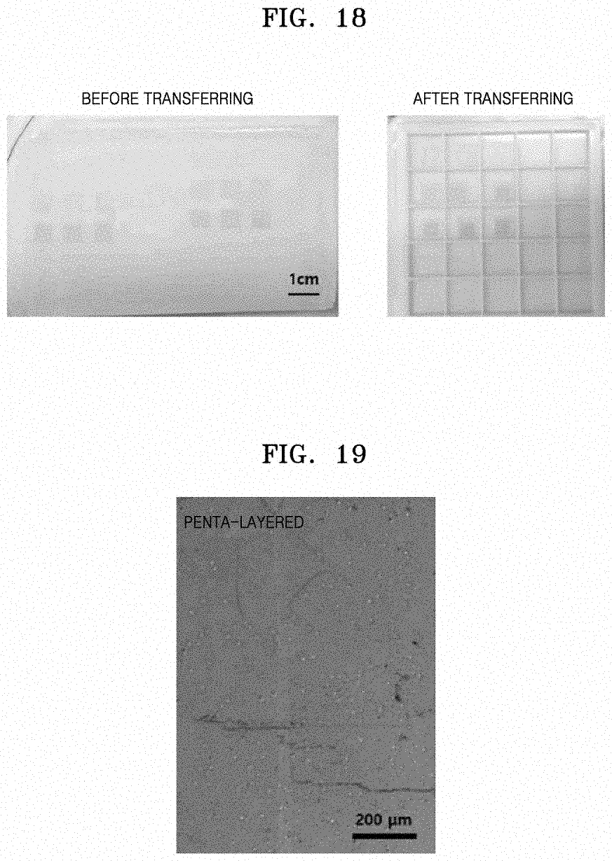

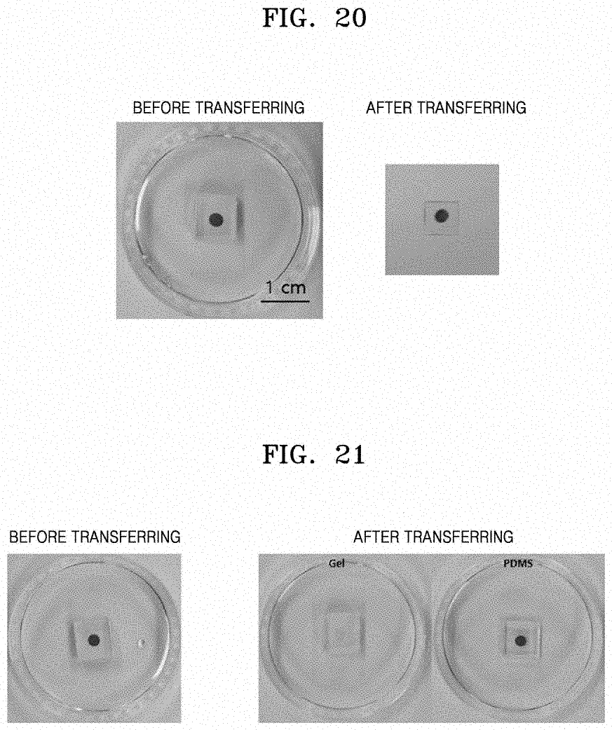

Each of the patterns formed on the hydrogel and prepared in Preparation Examples 6 and 7 was dried at room temperature for 30 minutes to be transferred to a final substrate. After the surface was dried, the printed pattern was transferred from the hydrogel to the substrate by contacting the substrate with the dried surface of the pattern and detaching the substrate therefrom. An image of a square pattern prepared in Preparation Example 6 and transferred to a quartz substrate is shown in FIG. 18. An image of a linear pattern prepared in Preparation Example 6 and transferred to a PET substrate is shown in FIG. 19. An image of a circular pattern prepared in Preparation Example 7 and transferred to the quartz substrate is shown in FIG. 20.

### Preparation Example 9 Preparation of Device by Molding Transfer Using Solution Capable of Hardening Pattern Prepared by Printing Process

Each of the ink solutions prepared according to Preparation Examples 3 to 5 was printed on the hydrogel prepared in Preparation Example 2 in multiple layers to prepare an electrode for analysis of characteristics. Subsequently, a polydimethylsiloxane solution was poured thereonto and hardened to transfer the printed pattern to a moldable polymer. An image of the pattern transferred by this method is shown in FIG. 21.

### Preparation Example 10 Preparation of Device by Floating Transfer of Pattern Formed by Printing Process

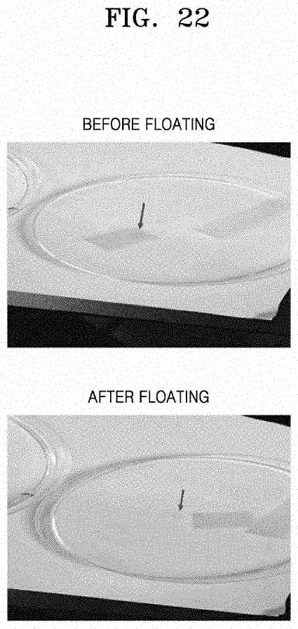

The pattern formed on the hydrogel and prepared in Preparation Example 6 was dried at room temperature for 30 minutes to be transferred to a final substrate. After the surface was dried, water was added thereto until the surface was immersed in water to separate the pattern from the surface of the hydrogel. Imagers before and after the separation are shown in FIG. 22.

### Preparation Example 11 Preparation of Biosensor Using Transferred Device

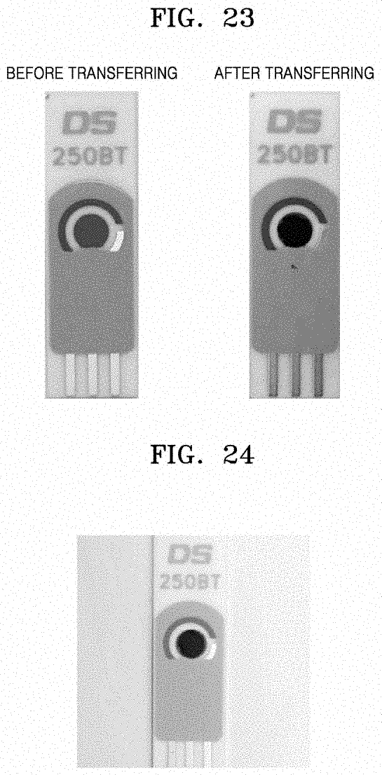

The ink solution prepared in Preparation Example 4 was printed on the hydrogel prepared in Preparation Example 1 by inkjet printing. The printed pattern was transferred to a commercial electrode (Manufactured by Dropsense, 250BT) according to the method described in Preparation Example 7, and an image thereof is shown in FIG. 23. After the electrode was transferred, 5 μL of a 5 w/v % polyethyleneimine (PEI) aqueous solution was dropped on a working electrode of the printed electrode and dried. After drying was completed, the excess PEI was washed away using distilled water. Subsequently, 5 μL of an aqueous solution of glucose oxidase (GOx) at a concentration of 100 mg/ml was additionally dropped on the working electrode and dried to prepare a 3rd-generation glucose sensor.

### Preparation Example 12 Preparation of All-printed Enzyme Electrode

The ink solution prepared in Preparation Example 4 was printed on the hydrogel prepared in Preparation Example 1 by inkjet printing. The hydrogel used as a substrate was immersed in water to remove a surfactant and other materials used to prepare the ink solution. The 5 w/v % polyethyleneimine (PEI) aqueous solution was printed on the printed electrode. By immersing the hydrogel in water, PEI was attached to the electrode by charge interaction, and the excess PEI was removed through the hydrogel substrate. A 25 mg/mL GOx aqueous solution was printed on the electrode. By immersing the hydrogel in water, GOx was attached to the electrode by charge interaction, and excess GOx was removed through the hydrogel substrate. The electrode was dried at room temperature for 30 minutes to be transferred to a final substrate. After the surface was dried, water was added thereto until the surface was immersed in water to separate the prepared electrode from the surface of the hydrogel. The separated electrode was transferred to a commercial electrode and an image thereof is shown in FIG. 24.

### Experimental Example

Evaluation of Electrical Characteristics of Pattern Prepared Using Ink Solution Including Carbonaceous Material with High Aspect Ratio

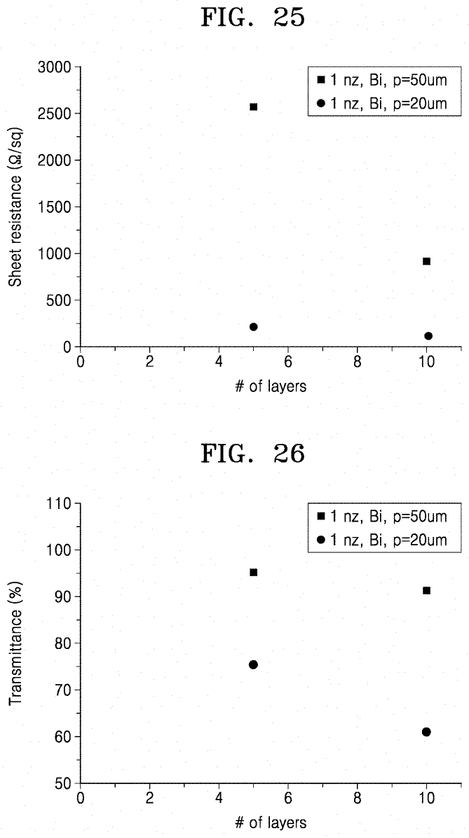

The ink solution prepared in Preparation Example 5 was printed 5 times or 10 times on the hydrogel prepared in Preparation Example 1 by inkjet printing. The printed pattern was contact transferred to a quartz substrate according to the method described in Preparation Example 8 to prepare a pattern for evaluation of electrical characteristics. Then, sheet resistance was measured by the van der Pauw method and the results are shown in FIG. 25.

As a result, as shown in FIG. 25, it was confirmed that sheet resistance decreases as the number of printing increases or a printing interval (p) is narrowed. That is, a low sheet resistance of 100 Ω/sq or less may be easily obtained.

Evaluation of Optical Characteristics of Pattern Formed Using Ink Solution Including Carbonaceous Material with High Aspect Ratio

The ink solution prepared in Preparation Example 5 was printed 5 times or 10 times on the hydrogel prepared in Preparation Example 1 by inkjet printing. The printed pattern was transferred to a quartz substrate according to the method described in Preparation Example 8 to prepare a pattern for evaluation of optical characteristics. Then, absorbance was measured in a wavelength range of 230 nm to 990 nm and converted using Equation 3 below to calculate transmittance, and the results are shown in FIG. 26.

Transmittance(%)=^{10-Absorbance(@ar 550 nm)} Equation 3

Measurement of Electrochemical Activity of GOx Enzyme Electrode Prepared Using Ink Solution Including Biomaterial

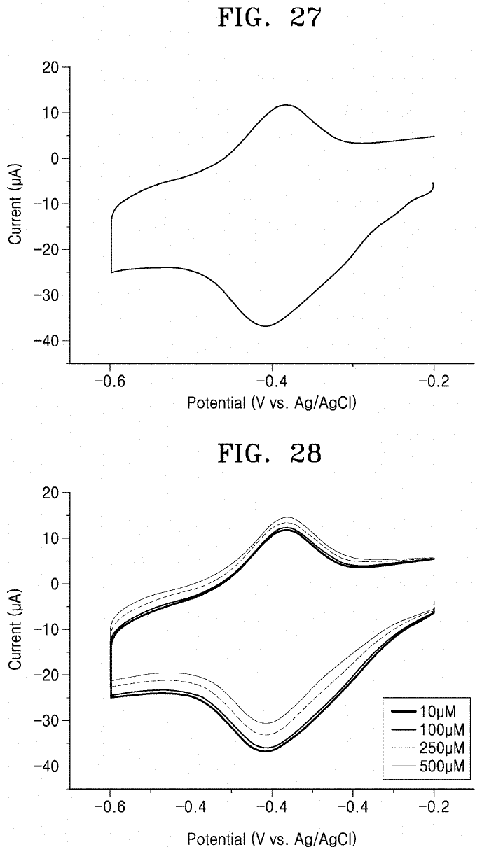

A negative voltage of −0.6 V to −0.2 V was applied to the glucose sensor prepared in Preparation Example 11 in 10 mM PBS buffer (pH=7.4, 79383, Sigma Aldrich) solution at a scan rate of 200 mV/s, and the results are shown in FIG. 27.

As shown in FIG. 27, the prepared glucose sensor showed strong redox peaks in a region of about −400 mV in cyclic voltammetry (CV) with respect to an Ag/AgCl reference electrode (3 M KCl saturated, PAR, K0260). These results indicate that an FAD redox center of GOx efficiently/directly formed electric pairs with the single-walled carbon nanotubes to cause a direct-electron-transfer (DET) as shown in a reaction scheme below.

FAD+2H++2e⁻->FADH₂

Based on this reaction, it may be seen that the enzyme present in the biosensor electrode prepared using a bio-adhesive may efficiently exchange electrons directly with the electrode.

Evaluation of Reactivity, to Glucose, of GOx Enzyme Electrode Prepared Using Ink Solution Including Biomaterial

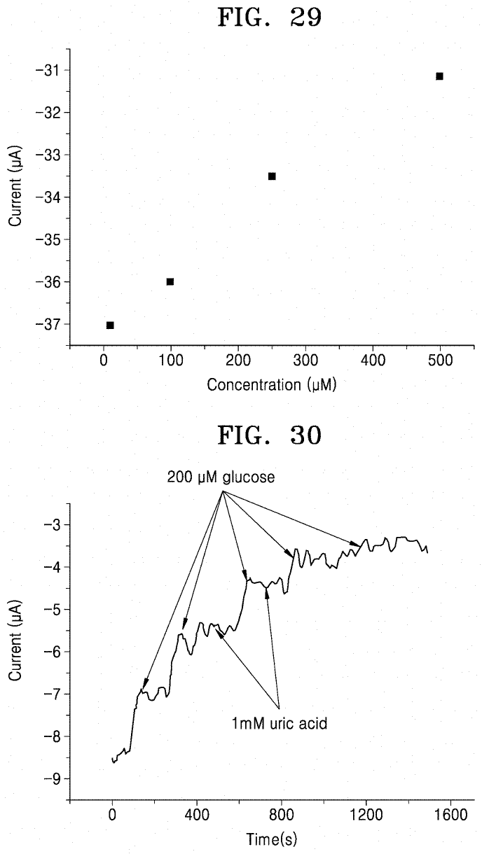

CV was performed while a voltage was applied to the glucose sensor prepared in Preparation Example 11 at a scan rate of 200 mV/s in 10 mM PBS buffer (pH=7.4, 79383, Sigma Aldrich) solution including 10 μM to 500 μM of glucose, and the results are shown in FIG. 28. Reduction currents were extracted in respective CV graphs and shown in FIG. 29.

As a result, as shown in FIG. 29, it was confirmed that the reduction currents increased linearly in the positive direction up to the glucose concentration of 500 μM as the concentration of glucose contained in the 10 mM PBS buffer increased while applying a voltage of −0.6 V to −0.2 V thereto. A sensitivity of the glucose sensor measured as described above was about 93.7 ρA/mM cm² or less. Based on the results, because the reduction current is linearly proportional to the glucose concentration with high sensitivity in a concentration range of 100 μM to 500 μM of glucose contained in non-invasively collectable body fluids (sweat, tear, saliva, etc.,) in the DET-based glucose sensor according to the present disclosure, the glucose sensor may be used as a DET-based 3^{rd}-generation wearable biosensor.

Evaluation of Real-time Monitoring Characteristics and Specificity of GOx Enzyme Electrode Prepared Using Ink Solution Including Biomaterial for Glucose

After applying a voltage of −0.4 V was applied to a working electrode of the GOx enzyme-based biosensor prepared in Preparation Example 11, and current flowing from each electrode was measured. Particularly, 200 μM glucose was injected 5 times in divided amounts, and 1 mM uric acid was injected twice in divided amounts. The results are shown in FIG. 30.

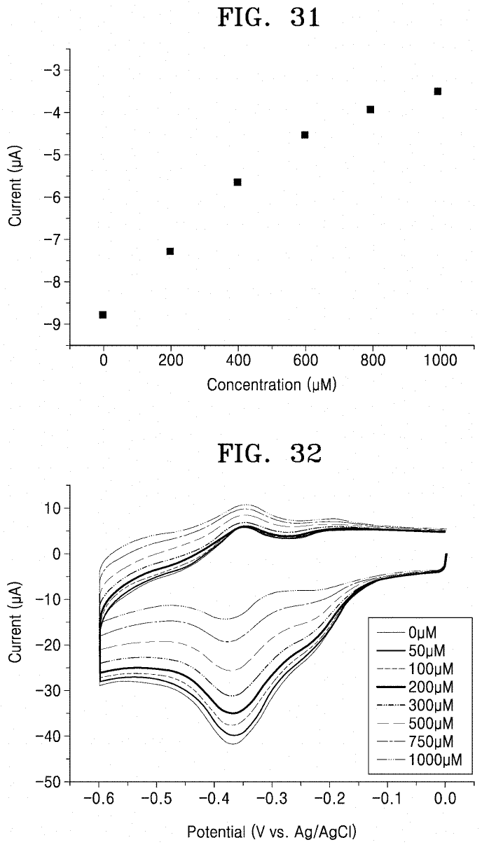

As a result, as shown in FIG. 30, an increase in current in a positive direction was observed in the GOx enzyme working electrode when glucose was added thereto. Changes in current with respect to glucose concentration are shown in FIG. 31. A measured sensitivity by real-time monitoring was about 52.8 pA/mM cm² or less. On the contrary, it was confirmed that the current did not change significantly when uric acid was injected thereinto. This allowed us to evaluate the specificity of the glucose sensor. As shown in FIG. 30, the 3rd-generation biosensor including the GOx enzyme electrode prepared according to an embodiment may operate in a non-invasively collectable body fluid such as saliva, tear, and sweat because the biosensor stably operates in an environment including not only the buffer solution of the 10 mM PBS solution but also interfering substances such as uric acid.

Evaluation of Reactivity and Specificity of All-printed Enzyme Electrode to Glucose

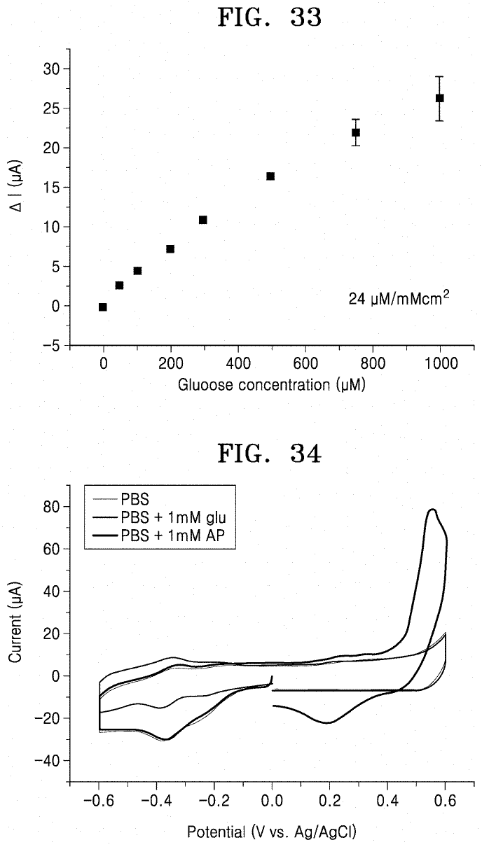

CV was performed while a voltage was applied to the enzyme electrode prepared in Preparation Example 12 at a scan rate of 200 mV/s in 10 mM PBS buffer (pH=7.4, 79383, Sigma Aldrich) solution including 0 μM to 1000 μM of glucose, and the results are shown in FIG. 32. Reduction currents were extracted in respective CV graphs and shown in FIG. 33.

As a result, as shown in FIG. 33, it was confirmed that the reduction currents increased linearly in the positive direction up to the glucose concentration of 1000 μM as the concentration of glucose contained in the 10 mM PBS buffer increased while applying a voltage of −0.6 V to 0 V. A sensitivity of the glucose sensor measured as described above was about 240 ρA/mM cm² or less.

CV was performed while a voltage was applied to the enzyme electrode prepared in Preparation Example 12 at a scan rate of 200 mV/s in each of 10 mM PBS buffer (pH=7.4, 79383, Sigma Aldrich) solution including 0 μM of glucose, 10 mM PBS buffer solution including 1000 μM of glucose, and 10 mM PBS buffer solution including 1000 μM of acetaminophene, and the results are shown in FIG. 34.

As a result, as shown in FIG. 34, it was confirmed that the reduction peak did not change significantly although acetaminophen was added thereto. Thus, high specificity of the enzyme electrode was confirmed.

Application of Nanoparticle Device Transferred to Polymer Glove to Produce Strain Sensor

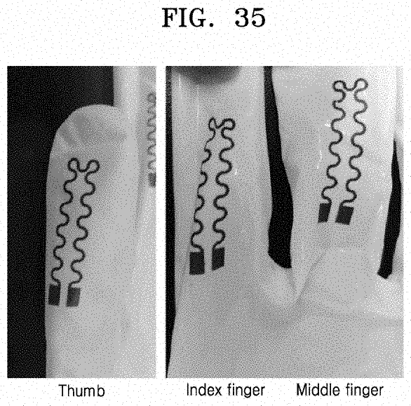

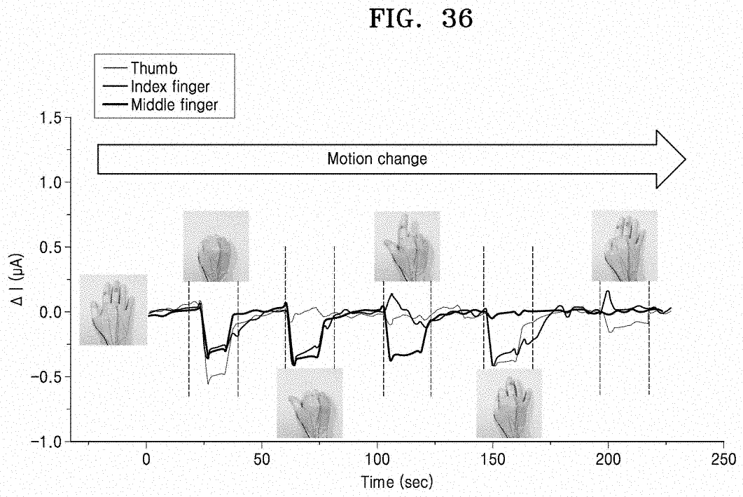

The ink solution prepared in Preparation Example 3 was printed on the hydrogel prepared in Preparation Example 1 by inkjet printing. The printed pattern was transferred to a polymer glove according to the method described in Preparation Example 8 to prepare a nanoparticle device, and the results are shown in FIG. 35. A glove to which the nanoparticle device is transferred was worn on a hand, and changes of resistance of the nanoparticle device corresponding to each finger was measured when each of the thumb, index finger, and middle finger was bent, and the results are shown in FIG. 36.

As a result, as shown in FIG. 36, it may be seen that resistance of the device corresponding to each finger changes in accordance with various hand motions. This allows us to confirm not only diversity of the substrate to which the nanoparticle device may be transferred but also excellent properties of the nanoparticle device.

What is claimed is:

1. A method of producing a nanoparticle device, the method comprising:
printing a colloidal composition on a hydrogel in a pattern, the colloidal composition comprising nanoparticles and a surfactant; and
forming a nanoparticle device by removing the surfactant comprised in the colloidal composition through pores inside the hydrogel.

2. The method of claim 1, wherein the surfactant is sodium cholate, sodium dodecyl sulfate, sodium deoxycholate, Nonidet P-40, Triton X-100, Tween 20®, polyethylene glycol 600, sodium lauryl sulfate, ammonium-oleate, cetyltrimethyl ammonium bromide, hydrolyzed tetraethyl orthosilicate, or any mixture thereof.

3. The method of claim 2, wherein the surfactant is sodium-cholate.

4. The method of claim 1, wherein the nanoparticles are graphene, highly oriented pyrolytic graphite (HOPG), graphene oxide, reduced graphene oxide, single-walled carbon nanotubes, double-walled carbon nanotubes, multi-walled carbon nanotubes, fullerene, metal nanowires, silver nanoparticles, platinum nanoparticles, gold nanoparticles, metal nanobeads, magnetic nanoparticles, silicon oxide, tungsten oxide, zinc oxide, neodymium oxide, titanium oxide, cerium oxide, iron oxide, boron nitride, titanium nitride, molybdenum disulfide (MoS₂), tungsten disulfide (WS₂), or any mixture thereof.

5. The method of claim 4, wherein the nanoparticles are graphene, single-walled carbon nanotubes, or any mixture thereof.

6. The method of claim 1, wherein the hydrogel is agarose gel, collagen, dextran, methyl cellulose, hyaluronic acid, polyethylene oxide, polyvinyl pyrrolidone, polyvinyl alcohol, sodium polyacrylate, acrylate polymer, acrylamide polymer, methacrylate polymer, or any mixture thereof.

7. The method of claim 1, wherein the pores inside the hydrogel have a diameter of 1 nm to 10 μm.

8. The method of claim 7, wherein the hydrogel is 0.1 wt % to 10 wt % agarose gel.

9. The method of claim 7, wherein the hydrogel is 0.1 wt % to 25 wt % acrylamide polymer.

10. The method of claim 1, wherein the composition further comprises a peptide having the ability to bind to a carbonaceous material or a phage displaying a peptide having the ability to bind to a carbonaceous material.

11. The method of claim 10, wherein the phage is M13 phage, F1 phage, Fd phage, If1 phage, Ike phage, Zj/Z phage, Ff phage, Xf phage, Pf1 phage, or Pf3 phage, each being genetically engineered to have the ability to bind to nanoparticles.

12. The method of claim 10, wherein the peptide has at least one amino acid sequence selected from SEQ ID NO: 1 to SEQ ID NO: 12.

13. The method of claim 1, wherein the pattern is a one-dimensional pattern, a two-dimensional pattern, a three-dimensional pattern, or any mixed pattern thereof.

14. The method of claim 1, wherein the printing of the colloidal composition in the pattern is performed by repeating, twice to 20 times, printing of the same colloidal composition or different colloidal compositions in multiple layers.

15. The method of claim 1, further comprising transferring the nanoparticle device formed on the hydrogel to a substrate.

16. The method of claim 15, wherein the transferring of the nanoparticle device to the substrate is performed by contacting the substrate with an upper surface of the hydrogel.

17. The method of claim 15, wherein the transferring of the nanoparticle device to the substrate is performed by pouring a solution capable of hardening onto the upper surface of the hydrogel, allowing the solution to harden, and then detaching the hardened solution from the hydrogel.

18. The method of claim 15, wherein the transferring of the nanoparticle device to the substrate comprises: separating the nanoparticle device from the hydrogel by adding a liquid in which the nanoparticle device formed on the hydrogel is able to float; and transferring the nanoparticle device to the substrate by contacting the substrate with a surface of the floating nanoparticle device facing the hydrogel.

19. The method of claim 18, wherein a multi-layer is formed by stacking another functional layer onto the composition or immobilizing an enzyme onto the composition, and the multi-layer is transferred to the substrate with the stacking order maintained.

20. The method of claim 1, wherein the nanoparticle device is a flexible electrode device, a transparent electrode device, a biosensor device, a strain sensor device, a pressure sensor device, a memory device, a logic device, an energy device, or an electrochemical device.

21. A composition for removing a surfactant dispersed in a nanoparticle aqueous solution,
the composition comprising a hydrogel having pores with a diameter of 1 nm to 10 μm.

22. A nanoparticle device produced according to the method of claim 1.
